SEQUENCE LISTING (24 sequences)

SEQ ID NO: 1 - protein, 8 aa, Artificial Sequence
  Zonulin antagonist
GRVCVQPG

SEQ ID NO: 2 - protein, 8 aa, Artificial Sequence
  Zonulin antagonist
GRVCVQDG

SEQ ID NO: 3 - protein, 8 aa, Artificial Sequence
  Zonulin antagonist
GRVLVQPG

SEQ ID NO: 4 - protein, 8 aa, Artificial Sequence
  Zonulin antagonist
GRVLVQDG

SEQ ID NO: 5 - protein, 8 aa, Artificial Sequence
  Zonulin antagonist
GRLCVQPG

SEQ ID NO: 6 - protein, 8 aa, Artificial Sequence
  Zonulin antagonist
GRLCVQDG

SEQ ID NO: 7 - protein, 8 aa, Artificial Sequence
  Zonulin antagonist
GRLLVQPG

SEQ ID NO: 8 - protein, 8 aa, Artificial Sequence
  Zonulin antagonist
GRLLVQDG

SEQ ID NO: 9 - protein, 8 aa, Artificial Sequence
  Zonulin antagonist
GRGCVQPG

SEQ ID NO: 10 - protein, 8 aa, Artificial Sequence
  Zonulin antagonist
GRGCVQDG

SEQ ID NO: 11 - protein, 8 aa, Artificial Sequence
  Zonulin antagonist
GRGLVQPG

SEQ ID NO: 12 - protein, 8 aa, Artificial Sequence
  Zonulin antagonist
GRGLVQDG

SEQ ID NO: 13 - protein, 8 aa, Artificial Sequence
  Zonulin antagonist
GGVCVQPG

SEQ ID NO: 14 - protein, 8 aa, Artificial Sequence
  Zonulin antagonist
GGVCVQDG

SEQ ID NO: 15 - protein, 8 aa, Artificial Sequence
  Zonulin antagonist
GGVLVQPG

SEQ ID NO: 16 - protein, 8 aa, Artificial Sequence
  Zonulin antagonist
GGVLVQDG

SEQ ID NO: 17 - protein, 8 aa, Artificial Sequence
  Zonulin antagonist
GGLCVQPG

SEQ ID NO: 18 - protein, 8 aa, Artificial Sequence
  Zonulin antagonist
GGLCVQDG

SEQ ID NO: 19 - protein, 8 aa, Artificial Sequence
  Zonulin antagonist
GGLLVQPG

SEQ ID NO: 20 - protein, 8 aa, Artificial Sequence
  Zonulin antagonist
GGLLVQDG

SEQ ID NO: 21 - protein, 8 aa, Artificial Sequence
  Zonulin antagonist
GGGCVQPG

SEQ ID NO: 22 - protein, 8 aa, Artificial Sequence
  Zonulin antagonist
GGGCVQDG

SEQ ID NO: 23 - protein, 8 aa, Artificial Sequence
  Zonulin antagonist
GGGLVQPG

SEQ ID NO: 24 - protein, 8 aa, Artificial Sequence
  Zonulin antagonist
GGGLVQDG

(19) United States
(12) Patent Application Publication
(10) Pub. No.: US 2016/0354426 A1
(43) Pub. Date: Dec. 8, 2016

(54) METHODS OF DIAGNOSING AND TREATING ASTHMA

(71) Applicant: Alba Therapeutics Corporation, Baltimore, MD (US)

(72) Inventor: Blake Paterson, Baltimore, MD (US)

(21) Appl. No.: 15/235,310

(22) Filed: Aug. 12, 2016

Related U.S. Application Data
(63) Continuation of application No. 14/971,663, filed on Dec. 16, 2015, now abandoned.
Application No. 14/971,663 is a continuation of application No. 12/513,336, filed on Feb. 18, 2010, now abandoned.
(60) Provisional application No. 60/856,380, filed on Nov. 3, 2006.

(51) Int. Cl. A61K 38/08; G01N 33/68; A61K 45/06

(52) U.S. Cl. CPC A61K 38/08; A61K 45/06; G01N 33/6893; G01N 2333/705; G01N 2800/122; G01N 2800/52

(57) ABSTRACT

The present invention provides materials and methods to diagnose asthma. In some embodiments, the present invention provides a method of diagnosing asthma by measuring the zonulin level of a subject. The present invention also provides methods and compositions for treating asthma that comprise one or more zonulin antagonist.

### FIELD OF THE INVENTION

The present invention relates to the fields of diagnostics and therapeutics. In particular the invention relates to diagnosing and treating asthma.

### BACKGROUND OF THE INVENTION

Mammalian epithelia contain structures referred to as zonula occludens (ZO) also referred to as tight junctions (TJs). These structures regulate the passage of materials through the epithelia by controlling access to the space between the epithelial cells (the paracellular pathway). To meet the many diverse physiological and pathological challenges to which epithelia are subjected, the tight junctions or zonula occludens must be capable of rapid, physiologic, reversible, transient, energy dependent, and coordinated responses that require the presence of a complex regulatory system. Examples of epithelia containing tight junctions include, but are not limited to, the intestines, (particularly the small intestine), the lungs, and the blood brain barrier.

In the absence of stimuli, the tight junctions are closed restricting access to the paracellular pathway. In the presence of stimuli, the tight junctions are reversibly opened. In U.S. Pat. Nos. 5,945,510 and 5,948,629, novel mammalian proteins that function as the physiological modulator of mammalian tight junctions, have been identified and purified. These mammalian proteins, referred to as “zonulin,” function as the physiological effector of mammalian tight junctions. Certain bacteria have been shown to have toxins that stimulate the opening of tight junctions. Vibrio cholerae infected with the filamentous bacteriophage CTXΦ, produces a toxin (zonula occludens toxin, ZOT) that has been shown to cause opening of tight junctions. It has been shown that 6 His-ΔG, an N-terminal deletion of ZOT in which the first 264 amino acids have been deleted and replaced with a six histidine purification tag, retains the ability to open tight junctions.

Intestinal tight junction dysfunction occurs in auto-immune diseases and in a variety of clinical conditions affecting the gastrointestinal tract, including food allergies, enteric infections, malabsorption syndromes such as celiac disease, and inflammatory bowel diseases. Healthy, mature gut mucosa with its intact tight junction serves as the main barrier to the passage of macromolecules. During the healthy state, small quantities of immunologically active proteins cross the gut host barrier. When the integrity of the tight junction system is compromised, as with prematurity or after exposure to radiation, chemotherapy, and/or toxins, a deleterious response to environmental antigens (including autoimmune diseases and food allergies) can occur.

Peptide antagonists of zonulin were described in U.S. Pat. No. 6,458,925, which is incorporated by reference herein in its entirety, which corresponds to WO 00/07609. Peptide antagonists of zonulin may bind to the ZOT receptor, yet not function to physiologically modulate the opening of mammalian tight junctions. The peptide antagonists competitively inhibit the binding of ZOT and zonulin to the ZOT receptor, thereby inhibiting the ability of ZOT and zonulin to physiologically modulate the opening of mammalian tight junctions.

Asthma is a chronic lung disorder that is marked by recurring episodes of airway obstruction (as from bronchospasm) manifested by labored breathing accompanied especially by wheezing and coughing and by a sense of constriction in the chest, and that is triggered by hyperreactivity to various stimuli (as allergens or rapid change in air temperature). In sensitized individuals, inhaled allergens (allergy triggers), such as pet dander, dust mites, cockroach allergens, molds, or pollens provoke a hyperimmune response characterized by recruitment of immune cells and production of IgE antibodies. Currently, asthma is generally treated using long term treatment with anti-inflammatories (e.g., glucocorticoids) and long-acting bronchodilators in combination with short term episodic treatment of acute attacks with short-acting bronchodilators. Glucocorticoids, the current gold standard treatment for allergic asthma, have been shown to act by suppressing the adaptive immune response (e.g., recruitment of inflammatory cells) while not suppressing innate immune response (e.g., epithelial barrier function and complement expression) (see Schleimer RP, Proc Am Thorac Soc. 2004;1(3):222-30. Glucocorticoids suppress inflammation but spare innate immune responses in airway epithelium.)

The barrier function of the epithelium is the first line of defense of the innate immune system. The epithelium secrets a variety of molecules (e.g., complement, collectins, lysozyme, and defensins) that lead to destruction of pathogens before they have an opportunity to penetrate. As discussed above, tight junctions play an integral role in maintaining and regulating the barrier function. Disruption of lung tight junction function of has been implicated in the development of allergic sensitization and asthma. In order for an allergen to reach antigen-presenting cells and induce an adaptive immune response, such as in asthma, the allergen must cross the lung epithelium. It has been shown that dust mite allergen Der p 1 causes disruption of lung tight junction structure and an increase in the permeability of lung epithelia. Wan, et al. J. Clinical Investigation 104(1):123-133 (1999). It was suggested that the transepithelial movement of Der p 1 may have been facilitated by the inherent proteolytic activity of Der p 1.

There remains a need in the art for methods and materials for the diagnosis and treatment of asthma. This need an others are met by the present invention.

### SUMMARY OF THE INVENTION

The present invention provides materials and methods for diagnosing asthma in a subject. In some embodiments, a method of diagnosing asthma in a subject may comprise obtaining a sample from the subject and determining zonulin in the sample, wherein the presence of zonulin is predictive of asthma. Any suitable sample may be used, for example, a bronchoalveolar lavage (BAL) sample. Such methods may also include determining one or more chemokines in the sample, for example, determining one or more of TARC, MDC, and IP-10.

The present invention also provides kits for diagnosing asthma. Such kits may comprise means for detecting zonulin. Such kits may also comprise means for detecting at least one cytokine. Means for detecting zonulin may comprise a first container containing a first antibody and a second container containing a second antibody. Kits of the invention may also comprise one or more compounds that may be used as control compounds to assess the activity of the reagents supplied in the kit. For example, to assess the activity of an antibody, the kits of the invention may comprise an antigen normally bound by the antibody. For example, a kit of the invention may comprise one or more containers containing ΔG fragment of zonula occludens toxin, ZOT, zonulin, TARC, MDC, or IP-10. In some embodiments, kits of the invention may comprise at least one antibody was raised against a protein comprising a fragment of zonula occludens toxin, for example, ΔG fragment of zonula occludens toxin. Kits of the invention may comprise antibodies raised against a protein comprising zonula occludens toxin. One or more antibodies used in kits of the invention may comprise one or more detectable moieties, e.g., biotin, fluorophores, chromophore, enzymes and the like.

Practice of some embodiments of the invention may require the determination of zonulin. Determining zonulin includes both detecting the presence or absence of zonulin in a sample as well as measuring the concentration of zonulin in a sample. In some embodiments of the invention, the concentration of zonulin may be measured while in other embodiments of the invention the presence or absence of zonulin may be assayed. Zonulin may be determined using any technique known to those of skill in the art. In some embodiments, determining zonulin may comprise contacting the sample with a first antibody that binds to zonulin under binding conditions, contacting the bound sample with a second antibody that binds zonulin under binding conditions, and detecting the presence of bound second antibody. Any first and second antibodies may used in the practice of the invention so long as they bind to both bind to zonulin with sufficient affinity to permit detection. In some embodiments, at least one antibody may be raised against a protein comprising a fragment of zonula occludens toxin. In one particular embodiment, the first antibody may be raised against a protein comprising a fragment of zonula occludens toxin, for example, the ΔG fragment of zonula occludens toxin. In some embodiments, at least one antibody may be raised against a protein comprising zonula occludens toxin. In a particular embodiment, the second antibody may be raised against a protein comprising zonula occludens toxin. Typically, the second antibody may comprise a detectable moiety, for example, biotin, fluorophores, chromophore, enzymes and the like.

In some embodiments, the present invention provides compositions and methods for preventing, ameliorating and/or treating asthma. In one embodiment, the present invention provides a method of preventing, ameliorating and/or treating asthma in a subject in need thereof, comprising contacting the subject with a composition comprising a tight junction antagonist (for example, a zonulin antagonist). Typically, the lung of the subject may be contacted with the tight junction antagonist. For example, a composition comprising a tight junction antagonist may be applied to the lung of a subject with asthma or susceptible to developing asthma. Suitable tight junction antagonists may be peptides and may comprise a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24. In a particular embodiment, the present invention provides a method of preventing, ameliorating and/or treating asthma comprising contacting the lung of a subject with a composition comprising a tight junction antagonist, wherein the antagonist comprises SEQ ID NO:15. Compositions for use in methods of the invention may also comprise one or more therapeutic agents.

In some embodiments, the present invention provides methods of monitoring the treatment of asthma in a subject. Such methods may comprise obtaining a first sample from the subject, determining zonulin level in the first sample, obtaining a second sample, determining zonulin level in the second sample, wherein a difference in zonulin level between the first sample and the second sample is indicative of a change in severity of asthma in the subject. In some embodiments, the zonulin level in the second sample may be lower than the zonulin level in the first sample and the change in level may indicate a reduction in severity of one or more asthma symptoms. In some embodiments, the zonulin level in the second sample may be higher than the zonulin level in the first sample and the change in level may indicate an increase in severity. Any suitable sample may be used so long as the presence of zonulin in the sample is related to the subject's asthma. For example, the samples may be bronchoalveolar lavage (BAL) samples and/or serum samples. Methods of the invention may further comprise determining one or more chemokines in the samples. Examples of cytokines that may be determined include, but are not limited to, one or more of TARC, MDC, and IP-10.

### BRIEF DESCRIPTION OF THE DRAWINGS

FIG. 1 is a micrograph of an in situ immunoflourescence microscopy of mouse lung sections incubated with either FITC-FZI/0 (right panel) or FITC-FZI/1 (left panel). Clusters of immunofluorescent FZI/0 particles were visualized at the level of distal airway tree, while no signal was detected in tissues incubated with FZI/1.

FIG. 2 shows the results of assays of the levels of TARC, MDC, and IP-10 measured by commercial ELISAs in serum and in BAL fluids obtained 20 hours after segmental allergen challenge of atopic subjects with saline or allergen (n=10; each colored dot represents a different subject). Short solid lines represent median values. Limits of detection are represented by dashed horizontal lines. P values are from the Mann-Whitney.

FIG. 3 shows the results of assays of levels of zonulin measured by sandwich ELISA in serum and in BAL fluids obtained 20 hours after segmental allergen challenge of atopic subjects with saline or allergen (n=4; each colored dot represents a different subject). Short solid lines represent median values.

### DETAILED DESCRIPTION OF THE INVENTION

As used herein a subject is any animal, e.g., mammal, upon which methods of the invention may be practiced and/or to which materials of the present invention may be administered. Subjects include, but are not limited to, humans.

As discussed above, in various embodiments, the present invention provides materials and methods for diagnosing asthma. Further, the present invention provides materials and methods for preventing, slowing the onset of, ameliorating and/or treating asthma in a subject in need thereof by, inter alia, administering to a subject in need of such preventing, slowing the onset of, ameliorating and/or treating, a pharmaceutically effective amount of a tight junction antagonist, for example, azonulin antagonist. Typically, antagonists suitable for use in the present invention bind to the zonula occludens toxin (ZOT) receptor, yet do not physiologically modulate the opening of mammalian tight junctions. In some embodiments, the antagonists may be peptides. The term “antagonist” is defined as a compound that that prevents, inhibits, reduces or reverses the response triggered by an agonist (i.e., zonulin). In one embodiment, the present invention provides materials and methods for preventing, slowing the onset of, ameliorating and/or treating asthma in a subject in need thereof by, inter alia, administering to a subject in need of such preventing, slowing the onset of, ameliorating and/or treating, a pharmaceutically effective amount of an antagonist of tight junction opening. In some embodiments, an antagonist may bind to the zonula occludens toxin (ZOT) receptor, yet not physiologically modulate the opening of mammalian tight junctions.

It has been shown that allergens can disrupt tight junctions in lung epithelium resulting in the introduction of the allergen to antigen presenting cells and that this might be an initial step in the development of asthma. Wan et al. supra. Without wishing to be bound by theory, the present invention provides evidence that transient disassembly of tight junctions occurs in asthma and is a zonulin-mediated event. Accordingly, the transient disassembly of tight junctions can be reduced and/or prevented by the administration of an antagonist of tight junction opening, for example, an antagonist of zonulin. Using the materials and methods of the present invention it is possible to prevent, slow the onset of, ameliorate and/or treat asthma by administering one or more compounds that prevent or reduce the transient disassembly of tight junctions in the lung epithelium. For example, zonulin is a normal physiological compound that enhances the permeability of anatomical barriers, for example, the lung epithelium, by mediating the opening of tight junctions. By administering a zonulin antagonist the permeability of the lung epithelium is maintained or decreased, thereby preventing, slowing the onset of ameliorating and/or treating asthma.

Antagonists of tight junction opening

As used herein, tight junction antagonists prevent, inhibit or reduce the opening of tight junctions, for example, the opening of tight junctions induced by a tight junction agonist. A tight junction antagonist may bind to the receptor that mediates tight junction agonist induced opening of tight junctions. Any antagonist of tight junction opening, for example, an antagonist of zonulin, may be used in the practice of the present invention. As used herein an antagonist of zonulin is any compound that bind to the zonulin receptor and that prevents, inhibits, reduces or reverses the response triggered by zonulin. For example, antagonists of the invention may comprise peptide antagonists. Examples of peptide antagonists include, but are not limited to, peptides that comprise an amino acid sequence selected from the group consisting of

(SEQ ID NO: 1)
Gly Arg Val Cys Val Gln Pro Gly,
(SEQ ID NO: 2)
Gly Arg Val Cys Val Gln Asp Gly,
(SEQ ID NO: 3)
Gly Arg Val Leu Val Gln Pro Gly,
(SEQ ID NO: 4)
Gly Arg Val Leu Val Gln Asp Gly,
(SEQ ID NO: 5)
Gly Arg Leu Cys Val Gln Pro Gly,
(SEQ ID NO: 6)
Gly Arg Leu Cys Val Gln Asp Gly,
(SEQ ID NO: 7)
Gly Arg Leu Leu Val Gln Pro Gly,
(SEQ ID NO: 8)
Gly Arg Leu Leu Val Gln Asp Gly,
(SEQ ID NO: 9)
Gly Arg Gly Cys Val Gln Pro Gly,
(SEQ ID NO: 10)
Gly Arg Gly Cys Val Gln Asp Gly,
(SEQ ID NO: 11)
Gly Arg Gly Leu Val Gln Pro Gly,
(SEQ ID NO: 12)
Gly Arg Gly Leu Val Gln Asp Gly,
(SEQ ID NO: 13)
Gly Gly Val Cys Val Gln Pro Gly,
(SEQ ID NO: 14)
Gly Gly Val Cys Val Gln Asp Gly,
(SEQ ID NO: 15)
Gly Gly Val Leu Val Gln Pro Gly,
(SEQ ID NO: 16)
Gly Gly Val Leu Val Gln Asp Gly,
(SEQ ID NO: 17)
Gly Gly Leu Cys Val Gln Pro Gly,
(SEQ ID NO: 18)
Gly Gly Lcu Cys Val Gln Asp Gly,
(SEQ ID NO: 19)
Gly Gly Leu Leu Val Gln Pro Gly,
(SEQ ID NO: 20)
Gly Gly Leu Leu Val Gln Asp Gly,
(SEQ ID NO: 21)
Gly Gly Gly Cys Val Gln Pro Gly,
(SEQ ID NO: 22)
Gly Gly Gly Cys Val Gln Asp Gly,
(SEQ ID NO: 23)
Gly Gly Gly Leu Val Gln Pro Gly,
and
(SEQ ID NO: 24)
Gly Gly Gly Leu Val Gln Asp Gly

When the antagonist is a peptide, any length of peptide may be used.

Generally, the size of the peptide antagonist will range from about 6 to about 100, from about 6 to about 90, from about 6 to about 80, from about 6 to about 70, from about 6 to about 60, from about 6 to about 50, from about 6 to about 40, from about 6 to about 30, from about 6 to about 25, from about 6 to about 20, from about 6 to about 15, from about 6 to about 14, from about 6 to about 13, from about 6 to about 12, from about 6 to about 11, from about 6 to about 10, from about 6 to about 9, or from about 6 to about 8 amino acids in length. Peptide antagonists of the invention may be from about 8 to about 100, from about 8 to about 90, from about 8 to about 80, from about 8 to about 70, from about 8 to about 60, from about 8 to about 50, from about 8 to about 40, from about 8 to about 30, from about 8 to about 25, from about 8 to about 20, from about 8 to about 15, from about 8 to about 14, from about 8 to about 13, from about 8 to about 12, from about 8 to about 11, or from about 8 to about 10 amino acids in length. Peptide antagonists of the invention may be from about 10 to about 100, from about 10 to about 90, from about 10 to about 80, from about 10 to about 70, from about 10 to about 60, from about 10 to about 50, from about 10 to about 40, from about 10 to about 30, from about 10 to about 25, from about 10 to about 20, from about 10 to about 15, from about 10 to about 14, from about 10 to about 13, or from about 10 to about 12 amino acids in length. Peptide antagonists of the invention may be from about 12 to about 100, from about 12 to about 90, from about 12 to about 80, from about 12 to about 70, from about 12 to about 60, from about 12 to about 50, from about 12 to about 40, from about 12 to about 30, from about 12 to about 25, from about 12 to about 20, from about 12 to about 15, or from about 12 to about 14 amino acids in length. Peptide antagonists of the invention may be from about 15 to about 100, from about 15 to about 90, from about 15 to about 80, from about 15 to about 70, from about 15 to about 60, from about 15 to about 50, from about 15 to about 40, from about 15 to about 30, from about 15 to about 25, from about 15 to about 20, from about 19 to about 15, from about 15 to about 18, or from about 17 to about 15 amino acids in length.

The peptide antagonists can be chemically synthesized and purified using well-known techniques, such as described in High Performance Liquid Chromatography of Peptides and Proteins: Separation Analysis and Conformation, Eds. Mant et al., C.R.C. Press (1991), and a peptide synthesizer, such as Symphony (Protein Technologies, Inc); or by using recombinant DNA techniques, i.e., where the nucleotide sequence encoding the peptide is inserted in an appropriate expression vector, e.g., an E. coli or yeast expression vector, expressed in the respective host cell, and purified therefrom using well-known techniques.

Therapeutic Agents

Compositions of the invention may comprise one or more therapeutic agents. Any therapeutic agent that is useful in the treatment of asthma may be used in conjunction with an antagonist of tight junction opening, for example, an antagonist of zonulin, in compositions of the invention. Examples of suitable therapeutic agents include, but are not limited to, medications that relieve asthma symptoms by relaxing muscles that have tightened around the airways. Other suitable therapeutic agents include medications that can prevent, reduce or reverse the swelling in the airways that causes asthma symptoms (e.g., anti-inflamatories). Other suitable therapeutic agents include long-acting bronchodilators that may be used together with anti-inflammatory medications. Examples of suitable medications include, but are not limited to, β-2-agonists, albuterol, metaproterenol sulfate, Combivent (a combination of a β-2-agonist and an anticholinergic), Duoneb (a combination of a β-2-agonist and an anticholinergic), Maxair, Tornalate, Ventolin, Xoponex, anticholinergics (such as ipratropium), anti-inflammatory drugs such as cromolyn and nedocromil, beclomethasone, budesonide (e.g., Pulmicort Turbuhaler), fluticasone (e.g., Flovent HFA), mometasone, corticosteroids, salmeterol, formoterol, Advair (a combination of a β-2-agonist and anti-inflammatory drug), Serevent, and Foradil.

Formulations

Compositions of the invention may formulated for pulmonary delivery (e.g., may be pulmonary dosage forms). Typically such compositions may be provided as pharmaceutical aerosols, which may be solution aerosols and/or powder aerosols. Those of skill in the art are aware of many different methods and devices for the formation of pharmaceutical aerosols, for example, those disclosed by Sciarra and Sciarra, Aerosols, in Remington: The Science and Practice of Pharmacy, 20th Ed., Chapter 50, Gennaro et al. Eds., Lippincott, Williams and Wilkins Publishing Co., (2000).

Typically, compositions comprising a tight junction antagonist (e.g., peptide antagonist) comprise a pharmaceutically effective amount of the antagonist. The pharmaceutically effective amount of antagonist (e.g., peptide antagonist) employed may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation.

In one embodiment, the dosage forms are in the form of a powder aerosol (i.e, comprise particles). These are particularly suitable for use in inhalation delivery systems. Powders may comprise particles of any size suitable for administration to the lung.

Powder formulations may optionally contain at least one particulate pharmaceutically acceptable carrier known to those of skill in the art. Examples of suitable pharmaceutical carriers include, but are not limited to, saccharides, including monosaccharides, disaccharides, polysaccharides and sugar alcohols such as arabinose, glucose, fructose, ribose, mannose, sucrose, trehalose, lactose, maltose, starches, dextran, mannitol or sorbitol. In one embodiment, a powder formulation may comprise lactose as a carrier.

Powder formulations may be contained in any container known to those in the art. Containers may be capsules of, for example, gelatin or plastic, or in blisters (e.g. of aluminum or plastic), for use in a dry powder inhalation device. In some embodiments, the total weight of the formulation in the container may be from about 5 mg to about 50 mg. In other embodiments, powder formulations may be contained in a reservoir in a multi-dose dry powder inhalation device adapted to deliver a suitable amount per actuation.

Powder formulations typically comprise small particles. Suitable particles can be prepared using any means known in the art, for example, by grinding in an airjet mill, ball mill or vibrator mill, sieving, microprecipitation, spray-drying, lyophilisation or controlled crystallisation. Typically, particles will be about 10 microns or less in diameter. Particles for use in the compositions of the invention may have a diameter of from about 0.1 microns to about 10 microns, from about 0.1 microns to about 9 microns, from about 0.1 microns to about 8 microns, from about 0.1 microns to about 7 microns, from about 0.1 microns to about 6 microns, from about 0.1 microns to about 5 microns, from about 0.1 microns to about 4 microns, from about 0.1 microns to about 3 microns, from about 0.1 microns to about 2 microns, from about 0.1 microns to about 1 micron, from about 0.1 microns to about 0.5 microns, from about 1 micron to about 10 microns, from about 1 micron to about 9 microns, from about 1 micron to about 8 microns, from about 1 micron to about 7 microns, from about 1 micron to about 6 microns, from about 1 micron to about 5 microns, from about 1 micron to about 4 microns, from about 1 micron to about 3 microns, from about 1 micron to about 2 microns, from about 2 microns to about 10 microns, from about 2 microns to about 9 microns, from about 2 microns to about 8 microns, from about 2 microns to about 7 microns, from about 2 microns to about 6 microns, from about 2 microns to about 5 microns, from about 2 microns to about 4 microns, or from about 2 microns to about 3 microns. In some embodiments, particles for use in the invention may be about 1 micron, about 2 microns, about 3 microns, about 4 microns, about 5 microns, about 6 microns, about 7 microns, about 8 microns, about 9 microns, or about 10 microns in diameter.

In another embodiment, the dosage forms are in the form of a solution aerosol (i.e., comprise droplets). Solution aerosols may be prepared using any means known to those of skill in the art, for example, an aerosol vial provided with a valve adapted to deliver a metered dose (e.g., 10 μl to 100 μ, e.g. 25 μl to 50 μl) of the composition Where the inhalable form of the active ingredient is a nebulizable aqueous, organic or aqueous/organic dispersion, the inhalation device may be a nebulizer, for example a conventional pneumatic nebulizer such as an airjet nebulizer, or an ultrasonic nebulizer, which may contain, for example, from 1 to 50 ml, commonly 1 to 10 ml, of the dispersion; or a hand-held nebulizer which allows smaller nebulized volumes, e.g. 10 μl to 100 μl. Typically, droplets will be about 10 microns or less in diameter. Particles and/or droplets for use in the compositions of the invention may have a diameter of from about 0.1 microns to about 10 microns, from about 0.1 microns to about 9 microns, from about 0.1 microns to about 8 microns, from about 0.1 microns to about 7 microns, from about 0.1 microns to about 6 microns, from about 0.1 microns to about 5 microns, from about 0.1 microns to about 4 microns, from about 0.1 microns to about 3 microns, from about 0.1 microns to about 2 microns, from about 0.1 microns to about 1 micron, from about 0.1 microns to about 0.5 microns, from about 1 micron to about 10 microns, from about 1 micron to about 9 microns, from about 1 micron to about 8 microns, from about 1 micron to about 7 microns, from about 1 micron to about 6 microns, from about 1 micron to about 5 microns, from about 1 micron to about 4 microns, from about 1 micron to about 3 microns, from about 1 micron to about 2 microns, from about 2 microns to about 10 microns, from about 2 microns to about 9 microns, from about 2 microns to about 8 microns, from about 2 microns to about 7 microns, from about 2 microns to about 6 microns, from about 2 microns to about 5 microns, from about 2 microns to about 4 microns, or from about 2 microns to about 3 microns. In some embodiments, particles and/or droplets for use in the invention may be about 1 micron, about 2 microns, about 3 microns, about 4 microns, about 5 microns, about 6 microns, about 7 microns, about 8 microns, about 9 microns, or about 10 microns in diameter.

Compositions of the invention may comprise one or tight junction antagonist at a level of from about 0.000001 wt % to about 50 wt %, from about 0.000001 wt % to about 45 wt %, from about 0.000001 wt % to about 40 wt %, from about 0.000001 wt % to about 35 wt %, from about 0.000001 wt % to about 30 wt %, from about 0.000001 wt % to about 25 wt %, from about 0.000001 wt % to about 20 wt %, from about 0.000001 wt % to about 15 wt %, from about 0.000001 wt % to about 10 wt %, from about 0.000001 wt % to about 5 wt %, from about 0.000001 wt % to about 2.5 wt %, from about 0.000001 wt % to about 1 wt %, from about 0.000001 wt % to about 0.1 wt %, from about 0.000001 wt % to about 0.01 wt %, from about 0.000001 wt % to about 0.001 wt %, from about 0.000001 wt % to about 0.0001 wt %, from about 0.000001 wt % to about 0.00005 wt %, from about 0.0001 wt % to about 50 wt %, from about 0.0001 wt % to about 45 wt %, from about 0.0001 wt % to about 40 wt %, from about 0.0001 wt % to about 35 wt %, from about 0.0001 wt % to about 30 wt %, from about 0.0001 wt % to about 25 wt %, from about 0.0001 wt % to about 20 wt %, from about 0.0001 wt % to about 15 wt %, from about 0.0001 wt % to about 10 wt %, from about 0.0001 wt % to about 5 wt %, from about 0.0001 wt % to about 2.5 wt %, from about 0.0001 wt % to about 1 wt %, from about 0.0001 wt % to about 0.1 wt %, from about 0.0001 wt % to about 0.01 wt %, from about 0.0001 wt % to about 0.001 wt %, from about 0.0001 wt % to about 0.0005 wt %, from about 0.1 wt % to about 50 wt %, from about 0.1 wt % to about 45 wt %, from about 0.1 wt % to about 40 wt %, from about 0.1 wt % to about 35 wt %, from about 0.1 wt % to about 30 wt %, from about 0.1 wt % to about 25 wt %, from about 0.1 wt % to about 20 wt %, from about 0.1 wt % to about 15 wt %, from about 0.1 wt % to about 10 wt %, from about 0.1 wt % to about 5 wt %, from about 0.1 wt % to about 2.5 wt %, from about 0.1 wt % to about 1 wt %, from about 0.1 wt % to about 0.5 wt %, from about 0.1 wt % to about 0.2 wt %, from about 1 wt % to about 50 wt %, from about 1 wt % to about 45 wt %, from about 1 wt % to about 40 wt %, from about 1 wt % to about 35 wt %, from about 1 wt % to about 30 wt %, from about 1 wt % to about 25 wt %, from about 1 wt % to about 20 wt %, from about 1 wt % to about 15 wt %, from about 1 wt % to about 10 wt %, from about 1 wt % to about 5 wt %, from about 1 wt % to about 2.5 wt %, from about 5 wt % to about 50 wt %, from about 5 wt % to about 45 wt %, from about 5 wt % to about 40 wt %, from about 5 wt % to about 35 wt %, from about 5 wt % to about 30 wt %, from about 5 wt % to about 25 wt %, from about 5 wt % to about 20 wt %, from about 5 wt % to about 15 wt %, from about 5 wt % to about 10 wt %, from about 5 wt % to about 9 wt %, from about 5 wt % to about 8 wt %, from about 5 wt % to about 7 wt %, or from about 5 wt % to about 6 wt % of the total weight of the composition. Compositions of the invention may comprise one or more tight junction antagonists at a level of about 0.00001 wt %, about 0.00005 wt %, about 0.0001 wt %, about 0.0005 wt %, about 0.001 wt %, about 0.005 wt %, about 0.01 wt %, about 0.05 wt %, about 0.1 wt %, about 0.5 wt %, about 1 wt %, about 5 wt %, about 10 wt %, about 15 wt %, about 20 wt %, about 25 wt %, about 30 wt %, about 35 wt %, about 40 wt %, about 45 wt %, or about 50 wt % based on the total weight of the composition.

Compositions of the invention may comprise one or more therapeutic agents at a concentration sufficient to cause the desired biological response (e.g., at a pharmaceutically effective concentration). Compositions of the invention may comprise one or therapeutic agents at a level of from about 0.1 wt % to about 50 wt %, from about 0.001 wt % to about 45 wt %, from about 0.001 wt % to about 40 wt %, from about 0.001 wt % to about 35 wt %, from about 0.001 wt % to about 30 wt %, from about 0.001 wt % to about 25 wt %, from about 0.001 wt % to about 20 wt %, from about 0.001 wt % to about 15 wt %, from about 0.001 wt % to about 10 wt %, from about 0.001 wt % to about 5 wt %, from about 0.001 wt % to about 2.5 wt %, from about 0.001 wt % to about 1 wt %, from about 0.001 wt % to about 0.5 wt %, from about 0.001 wt % to about 0.2 wt %, 0.01 wt % to about 50 wt %, from about 0.01 wt % to about 45 wt %, from about 0.01 wt % to about 40 wt %, from about 0.01 wt % to about 35 wt %, from about 0.01 wt % to about 30 wt %, from about 0.01 wt % to about 25 wt %, from about 0.01 wt % to about 20 wt %, from about 0.01 wt % to about 15 wt %, from about 0.01 wt % to about 10 wt %, from about 0.01 wt % to about 5 wt %, from about 0.01 wt % to about 2.5 wt %, from about 0.01 wt % to about 1 wt %, from about 0.01 wt % to about 0.5 wt %, from about 0.01 wt % to about 0.2 wt %, 0.1 wt % to about 50 wt %, from about 0.1 wt % to about 45 wt %, from about 0.1 wt % to about 40 wt %, from about 0.1 wt % to about 35 wt %, from about 0.1 wt % to about 30 wt %, from about 0.1 wt % to about 25 wt %, from about 0.1 wt % to about 20 wt %, from about 0.1 wt % to about 15 wt %, from about 0.1 wt % to about 10 wt %, from about 0.1 wt % to about 5 wt %, from about 0.1 wt % to about 2.5 wt %, from about 0.1 wt % to about 1 wt %, from about 0.1 wt % to about 0.5 wt %, from about 0.1 wt % to about 0.2 wt %, from about 1 wt % to about 50 wt %, from about 1 wt % to about 45 wt %, from about 1 wt % to about 40 wt %, from about 1 wt % to about 35 wt %, from about 1 wt % to about 30 wt %, from about 1 wt % to about 25 wt %, from about 1 wt % to about 20 wt %, from about 1 wt % to about 15 wt %, from about 1 wt % to about 10 wt %, from about 1 wt % to about 5 wt %, from about 1 wt % to about 2.5 wt %, from about 5 wt % to about 50 wt %, from about 5 wt % to about 45 wt %, from about 5 wt % to about 40 wt %, from about 5 wt % to about 35 wt %, from about 5 wt % to about 30 wt %, from about 5 wt % to about 25 wt %, from about 5 wt % to about 20 wt %, from about 5 wt % to about 15 wt %, from about 5 wt % to about 10 wt %, from about 5 wt % to about 9 wt %, from about 5 wt % to about 8 wt %, from about 5 wt % to about 7 wt %, or from about 5 wt % to about 6 wt % of the total weight of the composition. Compositions of the invention may comprise one or more therapeutic agents at a level of about 0.1 wt %, about 1 wt %, about 5 wt %, about 10 wt %, about 15 wt %, about 20 wt %, about 25 wt %, about 30 wt %, about 35 wt %, about 40 wt %, about 45 wt %, or about 50 wt % based on the total weight of the composition.

Compositions of the invention may comprise one or pharmaceutically acceptable excipients at a level of from about 0.1 wt % to about 50 wt %, from about 0.1 wt % to about 45 wt %, from about 0.1 wt % to about 40 wt %, from about 0.1 wt % to about 35 wt %, from about 0.1 wt % to about 30 wt %, from about 0.1 wt % to about 25 wt %, from about 0.1 wt % to about 20 wt %, from about 0.1 wt % to about 15 wt %, from about 0.1 wt % to about 10 wt %, from about 0.1 wt % to about 5 wt %, from about 0.1 wt % to about 2.5 wt %, from about 0.1 wt % to about 1 wt %, from about 0.1 wt % to about 0.5 wt %, from about 0.1 wt % to about 0.2 wt %, from about 1 wt % to about 50 wt %, from about 1 wt % to about 45 wt %, from about 1 wt % to about 40 wt %, from about 1 wt % to about 35 wt %, from about 1 wt % to about 30 wt %, from about 1 wt % to about 25 wt %, from about 1 wt % to about 20 wt %, from about 1 wt % to about 15 wt %, from about 1 wt % to about 10 wt %, from about 1 wt % to about 5 wt %, from about 1 wt % to about 2.5 wt %, from about 5 wt % to about 50 wt %, from about 5 wt % to about 45 wt %, from about 5 wt % to about 40 wt %, from about 5 wt % to about 35 wt %, from about 5 wt % to about 30 wt %, from about 5 wt % to about 25 wt %, from about 5 wt % to about 20 wt %, from about 5 wt % to about 15 wt %, from about 5 wt % to about 10 wt %, from about 5 wt % to about 9 wt %, from about 5 wt % to about 8 wt %, from about 5 wt % to about 7 wt %, or from about 5 wt % to about 6 wt % of the total weight of the composition. Compositions of the invention may comprise one or more pharmaceutically acceptable excipients at a level of about 0.1 wt %, about 1 wt %, about 5 wt %, about 10 wt %, about 15 wt %, about 20 wt %, about 25 wt %, about 30 wt %, about 35 wt %, about 40 wt %, about 45 wt %, or about 50 wt % based on the total weight of the composition.

Compositions of the invention may comprise one or more pharmaceutically-acceptable carriers. As used herein “pharmaceutically-acceptable carrier” includes any and all solvents, dispersion media, coatings, antibacterial and antifungal agents, isotonic and absorption delaying agents, and the like that are physiologically compatible. Pharmaceutically-acceptable carriers include, but are not limited to, sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Compositions of the invention may be formulated as a unit dose. A suitable unit dose of a peptide antagonist of the invention may be from about 20 μg to about 200 mg, from about 50 μg to about 200 mg, from about 100 μg to about 200 mg, from about 250 μg to about 200 mg, from about 500 μg to about 200 mg, from about 1 mg to about 200 mg, from about 2.5 mg to about 200 mg, from about 5 mg to about 200 mg, from about 10 mg to about 200 mg, from about 25 mg to about 200 mg, from about 50 mg to about 200 mg, or from about 100 mg to about 200 mg. For example, a suitable unit dose of peptide SEQ ID NO:15 may be from about 50 μg to about 50 mg. The precise amount of a unit dose will depend on the method of administration.

Methods of Use

The pharmaceutical compositions of the invention can be used for preventing, slowing the onset of, ameliorating and/or treating asthma. In one embodiment, the present invention provides a method of treating asthma by administering a composition comprising one or more tight junction antagonists, for example, one or more zonulin antagonists, and one or more therapeutic agents. Pharmaceutical compositions of the invention may be used to prevent asthma and/or to reduce the frequency and/or severity of asthmatic attacks.

In some embodiments, compositions of the invention may be given repeatedly over a protracted period, i.e., may be chronically administered. Typically, compositions may be administered one or more times each day in an amount suitable to prevent or reduce the likelihood of an asthma attack. Such pharmaceutical compositions may be administered chronically, for example, one or more times daily over a plurality of days. An amount suitable to prevent or reduce the likelihood of an asthma attack may be a unit dose as described above.

In some embodiments, pharmaceutical compositions of the invention may be used to treat acute asthmatic attacks. Typically, embodiments of this type will require administration of the pharmaceutical compositions of the invention to a subject undergoing an asthmatic attack in an amount suitable to reduce the severity of the attack. One or more administration may be used.

A composition according to the present invention may be pre-mixed prior to administration, or can be formed in vivo when two or more components (e.g., a tight junction antagonist and a therapeutic agent) are administered within 24 hours of each other. When administered separately, the components may be administered in either order (e.g. tight junction antagonist first followed by therapeutic agent or therapeutic agent first followed by tight junction antagonist). The components can be administered within a time span of about 12 hours, about 8 hours, about 4 hours, about 2 hours, about 1 hour, about 0.5 hour, about 0.25 hour, about 0.1 hour, about 1 minute, about 0.5 minute, or about 0.1 minute.

Administration of the compositions described above, e.g., compositions comprising one or more tight junction antagonists and optionally comprising one or more therapeutic agents, may be by inhalation. For example, one or more tight junction antagonists and one or more therapeutic agents or a mixture thereof, may be in inhalable form. An example of an inhalable form is an atomizable composition such as an aerosol comprising the tight junction antagonist, either alone or in combination with one or more therapeutic agents, in solution or dispersion in a propellant, or a nebulizable composition comprising a solution or dispersion of the active ingredient in an aqueous, organic or aqueous/organic medium. For example, the inhalable form of the compositions of the invention may be an aerosol comprising a mixture of one or more tight junction antagonists and one or more therapeutic agents in solution or dispersion in a propellant, or a combination of an aerosol containing one or more tight junction antagonists in solution or dispersion in a propellant with an aerosol containing one or more therapeutic agents in solution or dispersion in a propellant. In another example, the inhalable form of the compositions of the invention my be a nebulizable composition comprising a dispersion of one or more tight junction antagonists and one or more therapeutic agents in an aqueous, organic or aqueous/organic medium, or a combination of a dispersion of one or more tight junction antagonists with a dispersion of one or more therapeutic agents in such a medium.

The following examples are provided for illustrative purposes only, and are in no way intended to limit the scope of the present invention.

### EXAMPLE 1

It has been previously demonstrated that the zonulin pathway is operative in the respiratory tract and can be specifically activated for antigen delivery strategies (see Marinaro M, Di Tommaso A, Uzzau S, Fasano A, De Magistris MT. Infect Immun. 1999 Mar;67(3):1287-91.Zonula occludens toxin is a powerful mucosal adjuvant for intranasally delivered antigens and United States Patent Application 20060165722).

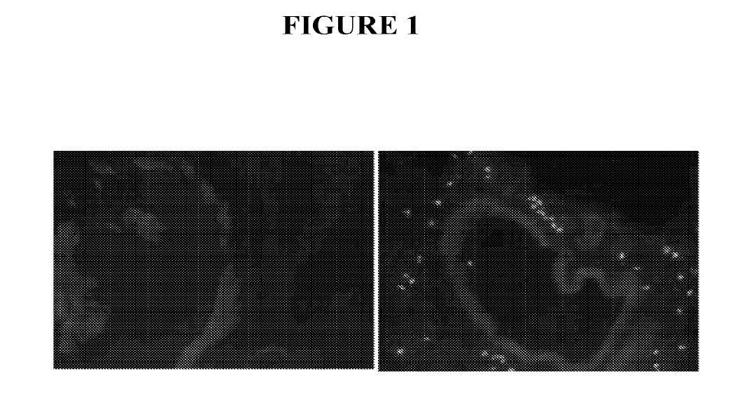

In situ immunofluorescence microscopy was used to establish the distribution of the zonulin receptor within the respiratory tract. Lung tissue sections (4 μm) made from frozen blocks were placed immediately on plain uncoated slides and incubated with either FITC-labeled FZI/0 (the zonulin synthetic peptide inhibitor that specifically binds to the zonulin receptor described in U.S. Pat. No. 6,458,925, Gly-Gly-Val-Leu-Val-Gln-Pro-Gly SEQ ID NO: 15) or FITC-labeled FZI/1 (a scrambled peptide described in U.S. Pat. No. 6,458,925, Val-Gly-Val-Leu-Gly-Arg-Pro-Gly SEQ ID NO: 25) The results are shown in FIG. 1. Cluster of immunofluorescence FZI/0 particles were visualized at the interface between endothelial (stained in red, FIG. 1) and epithelial layers, while no signal was detected in FZI/1-exposed tissues. These data suggest that zonulin receptors are present throughout the respiratory tract.

Human studies. Four subjects with both allergic rhinitis and asthma underwent segmental challenge with saline and antigen followed by bronchoalveolar lavage (BAL) as described by Bochner et al. (Bochner BS, Hudson SA, Xiao HQ, Liu MC. J Allergy Clin Immunol. 2003 Nov;112(5):930-4. Release of both CCR4-active and CXCR3-active chemokines during human allergic pulmonary late-phase reactions.) Briefly, for saline challenge, 5 mL of saline was instilled into 1 lung segment. For antigen challenge, 5 mL of low endotoxin ragweed or dust mite (Dermatophagoides pteronyssinus) antigen (Greer Laboratories, Lenoir, N.C.) at a concentration of 100 PNU/mL was instilled into another segment of the opposite lung. A second bronchoscopy was then performed 20 hours later with BAL to assess zonulin release and the inflammatory responses following both saline and allergen challenges. Blood was also obtained at the time of the second bronchoscopy. Cells were removed by centrifugation, and serum and BAL fluids were frozen at −80° C. and analyzed at a later date (29).

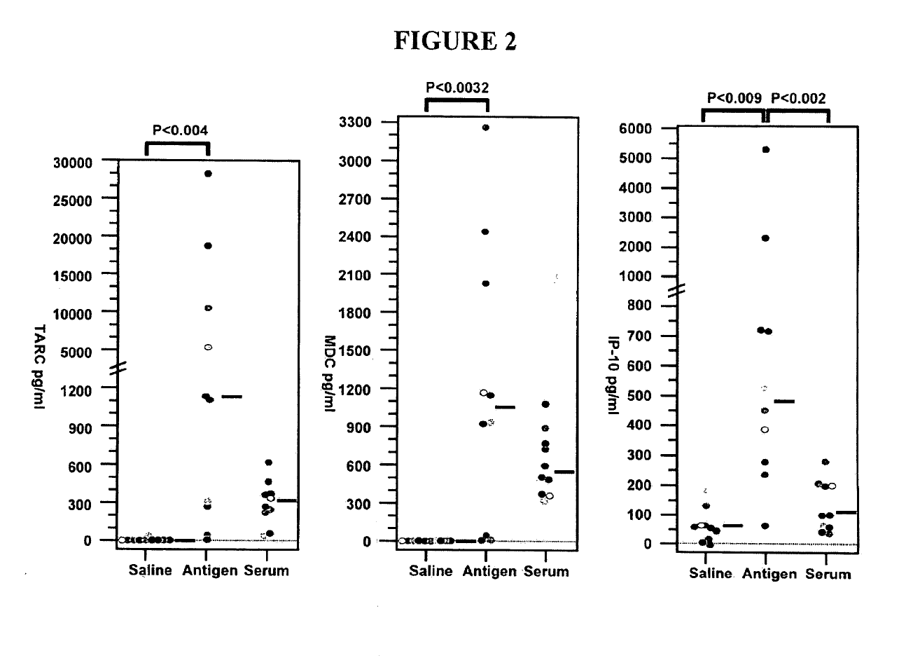

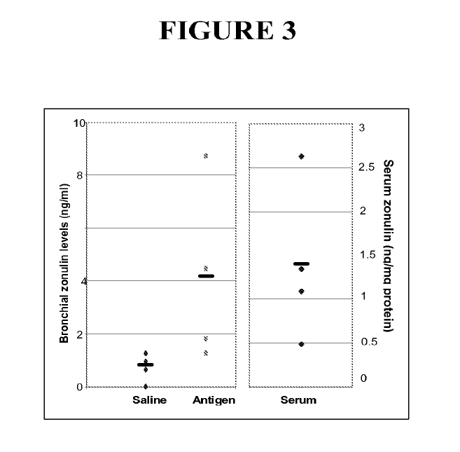

As expected, allergen challenge induced a vigorous inflammatory cellular response (REF). Shown in FIG. 2 are the ELISA results for the chemokines TARC (Thymus and activation-regulated chemokine), MDC (macrophage derived chemokine), and IP-10 (interferon-gamma-inducible protein 10) using BAL fluids and serum obtained 20 hours after segmental challenge. TARC and MDC levels were below the limit of detection in most of the samples from saline sites, but were detected in most BAL fluids from antigen-challenged sites. The same pattern was seen for IP-10, except that levels were detectable in BAL fluids at every saline site (median, 66 pg/mL). In comparing levels at antigen-challenged sites, TARC, MDC, and 1P-10 were significantly increased compared with those at saline sites. Interestingly, the release of chemokines in the airway lumen was paralleled by zonulin release that resulted six-fold higher in segments challenged with antigen as compared to those challenged with saline (FIG. 3, p<0.05).

While the invention has been described in detail, and with reference to specific embodiments thereof, it will be apparent to one of ordinary skill in the art that various changes and modifications can be made therein without departing from the spirit and scope thereof and such changes and modifications may be practiced within the scope of the appended claims. All patents and publications herein are incorporated by reference to the same extent as if each individual publication was specifically and individually indicated to be incorporated by reference in their entirety.

What is claimed is:

1. A method of diagnosing asthma in a subject, comprising:
obtaining a sample from the subject; and
determining zonulin in the sample, wherein the presence of zonulin is predictive of asthma.

2. A method according to claim 1, wherein the sample is a bronchoalveolar lavage (BAL) sample.

3. A method according to claim 1, further comprising determining one or more chemokines in the sample.

4. A method according to claim 3, wherein at least one of the chemokines is selected from the group consisting of TARC, MDC, and IP-10.

5. A method according to claim 1, wherein determining zonulin concentration comprises:
contacting the sample with a first antibody that binds to zonulin under binding conditions;
contacting the bound sample with a second antibody that binds zonulin under binding conditions; and
detecting the presence of bound second antibody.

6. A method according to claim 5, wherein at least one antibody was raised against a protein comprising a fragment of zonula occludens toxin.

7. A method according to claim 6, wherein the first antibody was raised against a protein comprising a fragment of zonula occludens toxin.

8. A method according to claim 6, wherein the first antibody was raised against a protein comprising AG fragment of zonula occludens toxin.

9. A method according to claim 6, wherein at least one antibody was raised against a protein comprising zonula occludens toxin.

10. A method according to claim 6, wherein the second antibody was raised against a protein comprising zonula occludens toxin.

11. A method according to claim 10, wherein the second antibody comprises a detectable moiety.

12. A method according to claim 11, wherein the detectable moiety comprises biotin.

13. A kit for diagnosing asthma, comprising:
means for detecting zonulin; and
means for detecting at least one cytokine

14. A kit according to claim 13, wherein the means for detecting zonulin comprises:
a first container containing a first antibody and a second container containing a second antibody.

15. A kit according to claim 13, further comprising a container containing AG fragment of zonula occludens toxin.

16. A kit according to claim 14, wherein at least one antibody was raised against a protein comprising a fragment of zonula occludens toxin.

17. A kit according to claim 14, wherein the first antibody was raised against a protein comprising a fragment of zonula occludens toxin.

18. A kit according to claim 14, wherein the first antibody was raised against a protein comprising AG fragment of zonula occludens toxin.

19. A kit according to claim 14, wherein at least one antibody was raised against a protein comprising zonula occludens toxin.

20. A kit according to claim 14, wherein the second antibody was raised against a protein comprising zonula occludens toxin.

21. A kit according to claim 14, wherein the second antibody comprises a detectable moiety.

22. A kit according to claim 21, wherein the detectable moiety comprises biotin.

23. A kit according to claim 13, further comprising a container selected from the group consisting of a container containing zonulin, a container contain the AG fragment of ZOT, a container containing TARC, a container containing MDC, and a container containing IP-10.

24. A kit according to claim 13, comprising means for detecting a chemokine selected from the group consisting of TARC, MDC, and IP-10.

25. A method for treating asthma in a subject in need thereof, comprising:
contacting the subject with a composition comprising a tight junction antagonist.

26. A method according to claim 25, wherein the antagonist comprises a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24.

27. A method according to claim 25, wherein the antagonist comprises SEQ ID NO:15.

28. A method according to claim 25, wherein the composition comprises a therapeutic agent.

29. A method according to claim 28, wherein the therapeutic agent is selected from a group consisting of muscle relaxers, bronchodilators and anti-inflamatories.

30. A method according to claim according to claim 28, wherein the therapeutic agent is selected from the group consisting of β-2-agonists, albuterol, metaproterenol sulfate, Combivent, Duoneb, Maxair, Tornalate, Ventolin, Xoponex, anticholinergics, ipratropium, cromolyn, nedocromil, beclomethasone, budesonide, fluticasone, mometasone, corticosteroids, salmeterol, formoterol, Advair, Serevent, and Foradil.

31. A method of monitoring the treatment of asthma in a subject, comprising:
obtaining a first sample from the subject; and
determining zonulin level in the first sample;
obtaining a second sample;
determining zonulin level in the second sample,
wherein a difference in zonulin level between the first sample and the second sample is indicative of a change in severity of asthma in the subject.

32. A method according to claim 31, wherein the zonulin level in the second sample is lower than the zonulin level in the first sample and the change in level indicates a reduction in severity.

33. A method according to claim 31, wherein the zonulin level in the second sample is higher than the zonulin level in the first sample and the change in level indicates an increase in severity.

34. A method according to claim 31, wherein the samples are bronchoalveolar lavage (BAL) samples.

35. A method according to claim 31, wherein the samples are serum samples.

36. A method according to claim 31, further comprising determining one or more chemokines in the samples.

37. A method according to claim 36, wherein at least one of the chemokines is selected from the group consisting of TARC, MDC, and IP-10.
